(19) United States
(12) Patent Application Publication
(10) Pub. No.: US 2008/0274943 A1
(43) Pub. Date: Nov. 6, 2008

(54) Process for Producing Narrow Range Alkoxylates

(75) Inventor: Joachim Karthauser, Sollentuna (SE)

Correspondence Address: PEARNE & GORDON LLP, 1801 EAST 9TH STREET, SUITE 1200, CLEVELAND, OH, 44114-3108, US

(73) Assignee: LINDE AG, Wiesbaden (DE)

(21) Appl. No.: 11/570,500

(22) PCT Filed: Jun. 16, 2005
(86) PCT No.: PCT/EP05/06468

§ 371 Date: Jan. 4, 2007

(30) Foreign Application Priority Data
Jun. 17, 2004 (EP) 04014231.7

(51) Int. Cl. C07C 41/03; C07C 43/11; C08K 3/00; C11D 1/72

(52) U.S. Cl. 510/506; 568/618; 568/678; 252/182.12

(57) ABSTRACT

According to the present invention there is provided a process for simultaneous or sequential separation of a mixture resulting from an alkoxylation reaction of a) at least one C4-C18 alcohol with b) at least one epoxide, comprising bringing said mixture into contact with pressurized CO2 having a concentration of at least 90% by weight, and collecting at least two fractions which differ in the average number of alkoxy units which are attached to said alcohol(s), at least a part of at least one but not all of said at least two fractions being recycled as raw material for the alkoxylation.

### TECHNICAL FIELD

The present invention relates to a process for the separation of a composition into at least two fractions whereby said composition comprises a mixture of C4-C8 alcohols which previously has been subjected to an alkoxylation reaction with epoxides, including ethylene oxide, propylene oxide or butylene oxide, or mixtures thereof, simultaneously or sequentially, said composition therefore containing a broad distribution of epoxide or alkoxy units, whereby at least a part of at least one but not all of the fractions is used as raw material for the alkoxylation. The present invention also relates to a product fraction obtained according to said process. The present invention further relates to the use of said product fraction.

### BACKGROUND ART

Non ionic surfactants are made by alkoxylation of alcohols, e.g. by base catalysed addition of ethylene oxide (EO) or propylene oxide (PO) to straight chain alcohols with 4 to 18 carbon atoms. These nonionic surfactants are mixtures of alcohols with a broad (Poisson-type) distribution of 0, 1, 2, 3, 4, 5 and so forth alkoxy units.

In terms of washing performance, certain fractions are more desired; often those fractions where the hydrophobic portion (the alkyl chain) and the hydrophilic portion (the alkoxy chain) are in a certain balance. Alcohols, with low alkoxylation degree are not water-soluble, alcohols with high alkoxylation degree are too water soluble to act as washing agent.

It is therefore desirable to offer alkoxylates with a narrow distribution of alkoxy groups.

Separations of liquids (olive oil, waxes etc.) using supercritical CO₂ is well known.

Separation of nonionic surfactants into fractions of low and high alkoxy content using solvents is not practiced industrially due to solvent handling issues and recovery costs. Instead, the approach in the industry has been to develop catalysts which enhance or favour the production of more narrow distributions.

The following abbreviations are used throughout the description and the claims:

RO=alcohol, e.g. fatty or synthetic C1-C20 alcohol
EO=ethylene oxide unit
PO=propylene oxide unit
BO=butylene oxide unit
(EO)3=three EO units chemically coupled
RO-(EO)x=alcohol chemically coupled with x 10 units, where x is a positive integer such as 1, 2, 3, 4, 5, 6, 7, 8, 9, 10, 11, 12, 13, 14, 15 . . . .

If x=0, RO-(EO)x designates a free alcohol.

The term ethoxylate is conveniently used in this text to denote all types of glycols and other alkoxylates.

C4-C18 alcohol as used in the description and in the claims should be interpreted as alcohols with 4 to 18 carbon atoms such as C4 alcohol, C5 alcohol, C6 alcohol, C7 alcohol, C8 alcohol, C9 alcohol, C10 alcohol, C11 alcohol, C12 alcohol, C13 alcohol, C14 alcohol, C15 alcohol, C16 alcohol, C17 alcohol, C18 alcohol.

CO₂ as used in the description and in the claims should be understood as a gas composition comprising mainly CO₂.

Nonionic surfactants are commercially available as broad distributions, comprising RO-(EO)x molecules with x being 0, 1, 2 and so forth. However, for a given process, e.g. washing in aqueous or CO₂ based systems, or as anti-freeze fluid or additive in textile processing or in the oil industry as drilling mud and in other applications, very often a specific ethoxylation degree and, in other words, a specific hydrophilic/lipophilic balance (HLB value) is desired.

Therefore, a problem with the prior art is that conventional production processes offer the desired molecules only as an average, and the desired product is diluted in significant amounts of material which either is too hydrophilic or too lipophilic for the desired function.

In addition, free alcohol and species with low alkoxylation degree create adverse smell in washing formulations, which is another incentive to minimize their concentrations.

Approaches to improve the product are the following: on laboratory scale, nonionics can be purified using various separation techniques, such as chromatography. Industrially, solvent extraction could be applied, however, handling and recovering solvents from foaming surfactants is expensive and not environment-friendly. Therefore, alkoxylate producers concentrate their efforts on developing catalysts for alkoxylation which favour narrow distributions of alkoxy units.

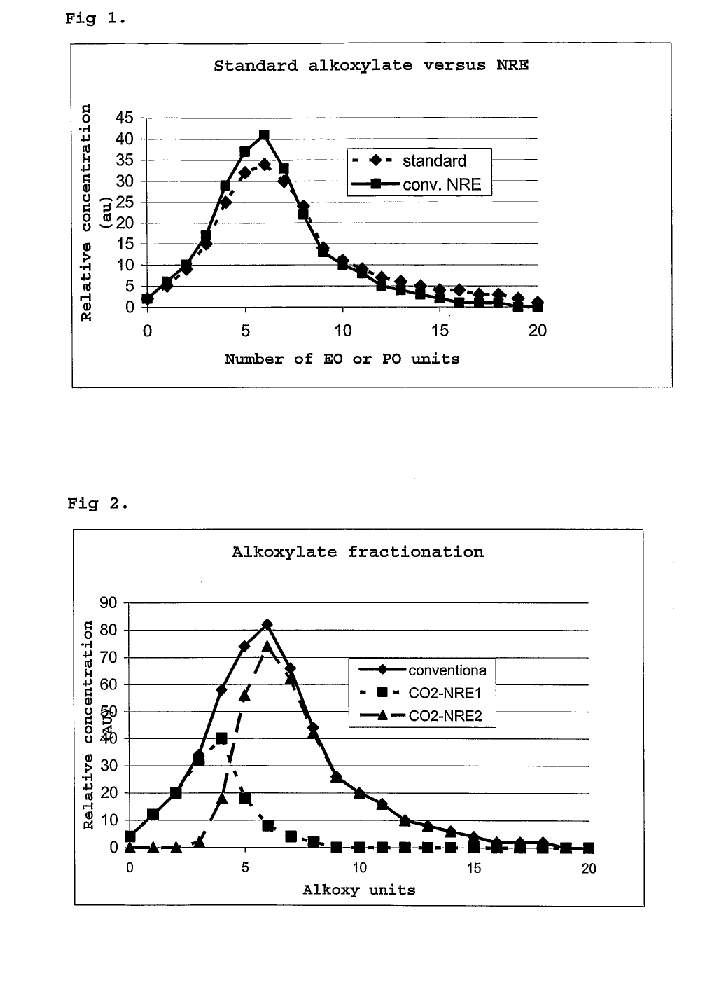

FIG. 1 shows typical distributions of standard ethoxylate versus conventional narrow range ethoxylate (conv. NRE). As can be seen, whilst the peak of the distribution is more pronounced for conventional narrow range ethoxylate (conv. NRE), the product still displays a quite broad distribution. FIG. 1 is a representation of the composition of industrially produced alkoxylates. For both grades, the peak of the distribution is at about 6 EO or PO units. The composition also contains free alcohol (zero EO units) and highly alkoxylated species with more than 10 EO units. The former (free alcohol) is hardly water-soluble; the latter species are hardly fat-soluble. Neither are good surfactants in a situation where the HLB value of RO-(EO)6 is specified.

The Japanese patents with publication numbers 01-193235 and 02-160738 both to Mitsubishi Heavy Ind Ltd Mitsubishi Petrochem Co Ltd, disclose a process to obtain aliphatic alcohol ethoxylate by extraction with CO₂ in a supercritical or pseudo-critical state as extracting agent. However it has turned out that this process is not economical because of the obtained side products such as free alcohols and species with low alkoxylation degree. It is usually not possible to find an economical outlet for these free alcohols and species with low alkoxylation degree. Under all circumstances, said separation generates significant amounts of side products such as free alcohol and alkoxylates low in EO content, and the production of narrow range alkoxylates will not be economic unless valuable outlets for the side products are found.

### SHORT DESCRIPTION OF THE INVENTION

It is an object of the present invention to provide a process for separation, which process solves the problem in the present state of the art, in particular the problem with undesired side products. The problem with unwanted side products is solved by recycling of free alcohols and fractions with high content of RO-(EO)x, with x being 1 or 2, i.e. the low molecular weight fraction. Thus the economy of the process is improved due to the minimization of the yield of undesired side products.

According to one aspect of the present invention there is provided a process for simultaneous or sequential separation of a mixture resulting from an alkoxylation reaction of a) at least one C4-C18 alcohol with b) at least one epoxide, comprising bringing said mixture into contact with pressurized CO₂ having a concentration of at least 90% by weight, and collecting at least two fractions which differ in the average number of alkoxy units which are attached to said alcohol(s), at least a part of at least one but not all of said at least two fractions being recycled as raw material for the alkoxylation.

According to an embodiment of the present invention there is provided a process wherein the epoxide b) is one or several selected from the group consisting of ethylene oxide, propylene oxide and butylene oxide.

There is also provided a process wherein the epoxide b) is ethylene oxide.

In the present invention there is provided a process, wherein at least a part of the fraction or fractions with the highest content(s) of free alcohols is/are recycled.

Also a process is provided, wherein at least a part of the fraction or fractions with the highest content(s) of RO-(EO)1 is/are recycled.

Also a process is provided, wherein at least a part of the fraction or fractions with the highest content(s) of RO-(EO)2 is/are recycled.

The present invention also provides a process, wherein said mixture is brought into contact with said CO₂ in counter-current mode.

There is provided a process, wherein said mixture is brought into contact with said CO₂ in an extraction column.

There is provided a process, wherein said mixture is brought into contact with said CO₂ in a falling film extractor.

There is provided a process, wherein said mixture is brought into contact with said CO₂ in a wiped film extractor.

According to another aspect of the present invention there is provided a product fraction obtained according to the process mentioned above.

According to another aspect of the present invention there is provided use of at least one fraction obtained according to the process mentioned above as improved surfactant, or as feedstock for improved surfactants.

According to one embodiment of the present invention there is provided use of at least one fraction mentioned above in aqueous cleaning processes.

There is also provided use of at least one fraction in cleaning processes using liquid and supercritical CO₂.

In the present invention there is provided use of at least one fraction in cleaning processes using organic solvents.

### DETAILED DESCRIPTION OF THE INVENTION

The present invention improves the separation of nonionic surfactants into fractions of low and high alkoxy content and thereby provides recycling of free alcohols and fractions with high content of RO-(EO)x, with x being 1 or 2 i.e. the low molecular weight fraction. This recycling drastically decreases the yield of free alcohols and fractions with high content of RO-(EO)x, with x being 1 or 2 i.e. the low molecular weight fraction and thereby improves the economy of the process.

The present invention relates to a process for separating a composition into at least two fractions whereby said composition comprises a mixture of C4-C18 alcohols which previously has been subjected to reaction with epoxides, including ethylene oxide, propylene oxide or butylene oxide, or mixtures thereof, simultaneously or sequentially, said composition therefore containing a broad distribution of epoxide or alkoxy units, wherein pressurized CO₂ in at least 90% concentration by weight is used as separation agent and said composition is separated in at least two fractions which differ in the average number of alkoxy units which are attached to said alcohols, and may differ in the average molecular weight of said alcohols. At least a part of at least one but not all of the fractions is used as raw material for alkoxylation. In a preferred embodiment of the present invention the fraction or fractions with a low alkoxylation degree and fraction or fractions comprising free alcohol are used as raw material for alkoxylation. By using this recycling of substances for the alkoxylation the undesired side products are used in an economic way and the entire process is more economic.

In alcoxylation, the reaction of the alcohol with the first EO (or epoxide) is usually rate-determining. Therefore, using a recycled alcoxylate with 1 or 2 or 3 epoxides already attached to the alcohol yields a more narrow EO distribution than a reaction with the free alcohol, which is another advantage of the present invention.

According to the present invention a composition of nonionic surfactants, as shown principally in FIG. 1, is subjected to an extraction with pressurized CO₂. The solvent CO₂ dissolves preferentially CO₂-philic species, and does not dissolve species which exhibit low solubility in CO₂. Therefore, by choosing temperature, pressure, time, and volume of solvent, a given composition can be split into two fractions with low content of alkoxy groups and high content of alkoxy groups respectively.

Technically, ethoxylates and propoxylates behave somewhat differently as propoxylates exhibit moderate CO₂ solubility, whereas ethoxylates are poorly soluble in CO₂. In general, an increase in molecular weight means reduced solubility, and the paraffin moiety is the main contributor to the absolute solubility of an alkoxylate. Therefore, CO₂ will preferentially dissolve species with high paraffin content, and the effect is the separation of a composition into two fractions as described in example 1 and 2 and depicted in FIG. 2. In the case of alkoxylates, the solubility of the alcohol moiety and the total chain length of the molecules are both significant factors, therefore the process results in the facile extraction of free alcohol and species with low EO content. Suitable devices for the extraction process are high-pressure counter-flow liquid-liquid extraction columns, as available from companies such as Uhde High Pressure Technologies of Germany, Natex of Austria and others. In a preferred embodiment of the present invention, the solvent CO₂ is recycled from the extracted fraction.

The employed solvent comprises mainly CO₂, however as the case may be; additives such as other gases (paraffins such as C3 to C5) or liquids (alcohols or suitable co-solvents which easily can be recovered from the product) may be employed. High pressure should be understood as a pressure being higher than 10 bar, and liquid CO₂ as well as supercritical CO₂ are included. The extraction temperature is case-dependant, but will be in the range of 10° C. up to 100° C.

For economic reasons, the process is preferably continuous, especially when performed in a large scale. However a person skilled in the art realises that the process also may be carried out batch-wise.

In one embodiment of the present invention the separation is performed in several steps to obtain several fractions with different alkoxylation degree. That is the process is carried out more than once, e.g. using a separated fraction, in order to further fractionate the product.

FIG. 2 shows the result of example 1 of the fractionation of a conventional alkoxylate composition into fractions with a narrower range of alkoxy units. The exact shape of the distributions of the fractions is a function of the process parameters, such as time, solvent volume, temperature, pressure and the like.

It will typically be desired to extract about 3-15% of the Original alkoxylate, and specifically as much as possible of the free alcohol (RO) and possibly RO-(EO)1, i.e. the low molecular weight fractions. At the same time it is desired, to minimize the recycle fraction for economic reasons, in other words, the extraction process shall be as selective as possible. The similarity of the molecules RO-(EO)x implies that the solubility differences between species are marginal which means constraints regarding the separation efficiency. The optimum conditions are different for different grades, however, as a rule of thumb working at the lower pressure end, i.e. close to or within the liquid CO₂ parameter room is advantageous as far as selectivity is concerned.

The present invention uses dense phase CO₂, liquid or supercritical CO₂, as selective solvents. The present invention allows an easy recovery of the solvent both from the low and high molecular weight stream. Specifically, the stream containing low molecular weight species has been found suitable as feedstock for further alkoxylation.

In general, narrow distributions of alkoxylates meet a given desired Performance specification, typically expressed as target HLB value, much better than a broad distribution. As alkoxylates are sold as many grades, e.g. C12-C15 alcohol with an average of 3, 5, 7 and 12 EO or PO units, a broad distribution with an average of e.g. 6 EO units can be split using the present invention into one fraction peaking at 4 EO units, and another fraction peaking at 7 EO units, as exemplified in example 1 and also shown in FIG. 2. The resulting fractions are less disperse, i.e. more active in the desired application.

The present invention can furthermore be used to separate the generally unwanted part of free alcohol or alcohols with one or two alkoxy units. In a preferred embodiment of the present invention said fraction is recycled to alkoxylation. In another embodiment of the present invention said fraction is used for other purposes, e.g. sulfonation.

A Stream of nonionics, e.g. fatty C4-C18 alcohols containing 1-20 alkoxy units is preferably led counter-current to a stream of pressurised CO₂, preferably supercritical CO₂ (sc-CO₂). CO₂ dissolves preferably alkoxylates containing low amounts of EO and PO respectively. The CO₂ stream enriched with low molecular weight alkoxylates is led to a distillation tank from where CO₂ is recovered by distillation, and the dissolved alkoxylates are recovered and transferred to a storage tank. From said tank the stream is fed into a standard alkoxylation reactor and is processed either separately or as component in a composition of alcohols. This recycling is particularly important in a large scale use of the present invention. The recycling minimizes the yield of undesired side products such as free alcohols and species with a low number of alkoxy units. The nonionics stream that has been extracted with CO₂ and thus has a lower concentration of the alkoxylates which were extracted by CO₂ (i.e. the narrow range main product) is led to a storage tank from which a remainder of CO₂ is recovered by pressure reduction; thereafter the nonionics stream is stored separately.

The preferred mode of operation is continuous in a falling film extractor or a wiped film extractor or another extractor containing at least one element which agitates the substrate which is to be extracted and thereby enhances the contact between said substrate and CO₂. Extraction in counter flow is preferred. A person skilled in the art realises that also other extraction modes may be used.

Preferred pressure ranges are 30-300 bar, preferably 40-130 bar, more preferably 40-100 bar.

Preferred temperature ranges are 10° C.-90° C., more preferably 15° C.-50° C.

Narrow range alkoxylates are desired compositions for cleaning also in non-aqueous media, such as textile cleaning and cleaning of industrial parts using liquid or supercritical CO₂. The present invention claims, apart from the process of separating alkoxylate streams into fractions, also the use of the fractions obtained as washing agents in both aqueous and non-aqueous cleaning, in particular in cleaning and, conditioning using dense phase CO₂. Regarding cleaning, an advantage of the present invention is that surfactants with good CO₂ solubility may be chosen. This will avoid the deposition of surfactants on the large total surface area provided by a batch of textiles, and in particular the deposition of unpleasantly smelling agents such as free alcohols is avoided. For the CO₂-based process of textile conditioning where deposition is the main purpose of the process, it is an even greater advantage that non-smelling agents are not present and therefore cannot precipitate on the textile.

Within the field of detergents, the concept can be used for a range of other surfactants, such as alkylpolyglucosides, non-ylphenol-based surfactants, fatty acid derivatives and the like.

### EXAMPLE 1

A batch-extraction was carried out, aiming at separation of a mixture comprising a broad distribution of about 2% free alcohol RO, 5% RO-(EO)1, 8% RO-(EO)2, etc. with a peak at RO-(EO)6 (approx. 20%), and a decreasing concentration up to RO-(ED)12 (about 3%) and higher homologues.

Supercritical CO₂ was continuously passed through a separation-column with reflux. This column was 3 m high, had a volume of 3 litres and an effective packing of 2.1 m. Within the current configuration the column contained about 10-12 theoretical plates. The reflux was induced by means of a heating coil. This coil heated the supercritical CO₂ at the top of the column resulting in a density reduction of the CO₂ and a lower dissolution power of the CO₂.

The column was loaded with 128 g of the original mixture described above. This original mixture was previously heated to 28° C. to entirely melt the mixture. After about 30 min and gentle pressurisation to 130 bar the extraction was started by flowing the pressurised and heated (T=40° C.) CO₂ through the heated column while the heating coil was switched on resulting in a temperature at the top of about T^{top}=53° C. At the top of the additionally heated column the dissolved compound partially condensed and flowed back on the packing. The still dissolved compounds passed on to the separator (at 50 bar and about 20° C.) where they precipitated. Different fractions were obtained by regularly emptying the separator.

From the first experiment eight fractions were obtained during the experiment. Most remarkable was the strong soap-like odour of in particular fraction number 1, which seemed to have a higher melting point than all other fractions.

For the first 13 wt % of the original mixture, about 3 kg CO₂ were passed through the extraction column (p=130 bar, T=40° C.) while the average CO₂-density at the top was 580 g/m³ at T=55° C.). As the experiment was carried out in batch mode, some 16% of the starting material was not recovered directly, this (high molecular weight) fraction is not included in the analysis described below (see FIG. 2).

Two representative fractions “CO2-NRE1” and “CO2-NRE2” are shown in FIG. 2 and show the narrow range of the distribution of alkoxy units which is obtained using this technique. In FIG. 2 the fractions according to the present invention are compared to conventional narrow range ethoxylates “conventiona”.

In a continuous process according to the present invention the fractions with high concentration of tree alcohol and high concentration of RO-(EO)1 and RO-(EO)2 and would have been recycled back to the alkoxylation reaction leading to the mixture to be separated, and thus the yield of these undesired side products would have been minimized.

### EXAMPLE 2

Batch-extractions were carried out in the same column and in the same way as in example 1. The column was loaded with 122 g of the original mixture described above. This original mixture was previously heated to 26° C. to entirely melt the mixture. After about 30 min and gentle pressurisation to 100 bar the extraction was started by flowing the pressurised and heated (T=40° C.) CO₂ through the heated column while the heating coil was switched on resulting in a temperature at the top of about T^{top}=53° C. At the top of the additionally heated column the dissolved compound partially condenses and flows back on the packing. The still dissolved compounds passed on to the separator (at 50 bar and about 20° C.) where they precipitated. Different fractions were obtained by regularly emptying the separator.

The second experiment resulted in six fractions with increasing average EO content. The first sample was characterized by a strong soap-like odour and a yellowish colour.

For the first 13 wt % of the original mixture, about 6 kg CO₂ were passed through the extraction column (p=100 bar, T=40° C.) while the average CO₂-density at the top was 330 g/m³ at T=55° C.). A mass balance for the 2^{nd} experiment was not carried out.

Even though the present invention has been exemplified by the two examples above, the scope of the invention should not be construed to be limited to those examples. The scope of the present invention is defined by the claims.

1. A process for simultaneous or sequential separation of a mixture resulting from an alkoxylation reaction of a) at least one C4-C18 alcohol with b) at least one epoxide, comprising bringing said mixture into contact with pressurized CO₂ having a concentration of at least 90% by weight, and collecting at least two fractions which differ in the average number of alkoxy units which are attached to said alcohol(s), at least a part of at least one but not all of said at least two fractions being recycled as raw material for the alkoxylation.

2. A process according to claim 1, wherein the epoxide b) is one or several selected from the group consisting of ethylene oxide, propylene oxide and butylene oxide.

3. A process according to claim 1 wherein the epoxide b) is ethylene oxide.

4. A process according to any of claims 1-3, wherein at least a part of the fraction or fractions with the highest content(s) of free alcohols is/are recycled.

5. A process according to any of claims 1-4, wherein at least a part of the fraction or fractions with the highest content(s) of RO-(EO)1 is/are recycled.

6. A process according to any of claims 1-5, wherein at least a part of the fraction or fractions with the highest content(s) of RO-(EO)2 is/are recycled.

7. A process according to any of claims 1-6, wherein said mixture is brought into contact with said CO₂ in mode.

8. A process according to any of claims 1-7, wherein said mixture is brought into contact with said CO₂ in an extraction column.

9. A process according to any of claims 1-7, wherein said mixture is brought into contact with said CO₂ in a falling film extractor.

10. A process according to any of claims 1-7, wherein said mixture is brought into contact with said CO₂ in a wiped film extractor.

11. Product fraction obtained according to the process of any of claims 1-10.

12. Use of at least one fraction obtained according to the process according to any of claims 1-10 as improved surfactant, or as feedstock for improved surfactants.

13. Use of at least one fraction according to any of claims 1-10 in aqueous cleaning processes.

14. Use of at least one fraction according to any of claims 1-10 in cleaning processes using liquid and supercritical CO₂.

15. Use of at least one fraction according to any of claims 1-10 in cleaning processes using organic solvents.
